(19) United States
(12) Patent Application Publication
(10) Pub. No.: US 2015/0352352 A1
(43) Pub. Date: Dec. 10, 2015

(54) FLEX SAFE SUTURE SLEEVE

(71) Applicant: Cardiac Pacemakers, Inc., St. Paul, MN (US)

(72) Inventors: Brian Soltis, St. Paul, MN (US); Andrew L. De Kock, Andover, MN (US)

(21) Appl. No.: 14/708,714

(22) Filed: May 11, 2015

Related U.S. Application Data
(60) Provisional application No. 62/008,310, filed on Jun. 5, 2014.

(51) Int. Cl. A61N 1/05; B29C 45/00; B29C 47/00

(52) U.S. Cl. CPC A61N 1/0558; B29C 47/0038; B29C 45/0055; B29K 2083/00

(57) ABSTRACT

A suture sleeve for securing an implantable lead with a suture includes an elongate, tubular body having a proximal end portion with a proximal face, a distal end portion with a distal face, and an exterior surface. The suture sleeve also includes a lumen within the tubular body extending from the proximal end portion to the distal end portion, wherein the lumen is sized to receive the implantable lead. The suture sleeve also includes a nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control the radius of curvature of the implantable lead that bends along the inner transition region.

This application claims priority to Provisional Application No. 62/008,310, filed Jun. 5, 2014, which is herein incorporated by reference in its entirety.

### TECHNICAL FIELD

The present disclosure relates generally to implantable medical leads. More specifically, the present disclosure relates to suture sleeves for anchoring medical leads to tissue of a patient.

### BACKGROUND

Medical leads are secured to a patient's tissue in a variety of applications using anchoring devices, including those commonly referred to as suture sleeves. For example, in many applications, an electrical lead connected to a neural stimulation modulation or a cardiac rhythm management (CRM) device, such as a pacemaker, is secured to patient tissue at a vein entry site or other location to help prevent both acute and chronic lead migration and dislodgement. In particular, the leads are anchored in place by securing a suture sleeve about the insulation of the lead and suturing the suture sleeve to the patient's tissue.

### SUMMARY

Disclosed herein are various embodiments of suture sleeves and methods for making suture sleeves.

In Example 1, a suture sleeve for securing an implantable lead with a suture includes an elongate, tubular body having a proximal end portion with a proximal face, a distal end portion with a distal face, and an exterior surface. The suture sleeve also includes a lumen within the tubular body extending from the proximal end portion to the distal end portion, wherein the lumen is sized to receive the implantable lead. The suture sleeve also includes a nonlinear, inner transition region extending from the lumen to at least one of the proximal face and the distal face of the tubular body, wherein the inner transition region is adapted to control the radius of curvature of the implantable lead that bends along the inner transition region.

In Example 2, the suture sleeve according to Example 1, wherein the suture sleeve further comprises an outer transition region that extends between the exterior surface of the suture sleeve and at least one of the proximal face and the distal face of the suture sleeve.

In Example 3, the suture sleeve according to either Example 1 or 2, wherein the inner transition region includes at least one curvilinear surface.

In Example 4, the suture sleeve according to any of Examples 1-3, wherein the inner transition region includes at least one chamfered edge, and wherein each chamfered edge has an obtuse angle between adjacent surfaces thereof.

In Example 5, the suture sleeve according to any of Examples 1-4, wherein the suture sleeve further comprises the inner transition region extending from the lumen to the proximal face of the tubular body.

In Example 6, the suture sleeve according to any of Examples 1-5, wherein the suture sleeve further comprises the inner transition region extending from the lumen to the distal face of the tubular body.

In Example 7, the suture sleeve according to Example 6, wherein the suture sleeve further comprises a second inner transition region extending from the lumen to the proximal end of the tubular body.

In Example 8, the suture sleeve according to any of Examples 1-7, wherein the suture sleeve further comprises the inner transition region that controls bending of the lead to a radius of curvature in a range of about 0.010 inches to 0.250 inches.

In Example 9, the suture sleeve according to any of Examples 1-8, wherein the suture sleeve further comprises at least a portion of the tubular body made of a first material and at least a portion of the tubular body that includes the inner transition region made of a second material having a higher durometer than the first material.

In Example 10, the suture sleeve according to Example 9, wherein the first material comprises silicone or polyurethane.

In Example 11, the suture sleeve according to Example 10, wherein the second material comprises polyurethane, tecothane, polyether ether ketone (PEEK), stainless steel, MP35N, titanium or platinum.

In Example 12, a method of making a suture sleeve for securing an implantable lead includes forming a tubular body having a proximal end portion with a proximal face, a distal end portion with a distal face, an exterior surface, a lumen sized to receive the implantable lead, and an inner surface within the lumen. The method also includes forming a nonlinear, inner transition region extending from the lumen to at least one of the proximal face and the distal face of the tubular body, wherein the inner transition region is adapted to control a radius of curvature of the implantable lead that bends along the inner transition region.

In Example 13, the method according to Example 12, wherein forming a tubular body includes forming a channel around the circumference of the tubular body, wherein the channel is sized to accommodate a suture within the channel.

In Example 14, the method according to Example 12 or 13, the inner transition region includes at least one curvilinear surface or at least one chamfered edge having an obtuse angle between adjacent surfaces thereof.

In Example 15, the method according to according to any of Examples 12-14, wherein forming the inner transition region comprises using one of an injection molding, extrusion, sanding or a machining process.

In Example 16, an implantable system comprising a pulse generator, an implantable medical electrical lead having a lead body with a proximal end and a distal end, the proximal end coupled to the pulse generator, a suture sleeve for securing an implantable lead with a suture, and at least one anchoring element configured to be secured circumferentially about the tubular body to secure the suture sleeve at particular location within the patient's body. The suture sleeve includes an elongate, tubular body having a proximal end portion with a proximal face, a distal end portion with a distal face, and an exterior surface. The suture sleeve also includes a lumen having an inner surface that is within the tubular body and extends from the proximal end portion to the distal end portion, wherein the lumen is sized to receive the implantable lead. The suture sleeve also includes a non-linear, inner transition region extending from the lumen to at least one of the proximal face and the distal face of the tubular body, the inner transition region being a curvilinear surface or a chamfered edge having an obtuse angle between adjacent surfaces thereof, wherein the inner transition region is adapted to control a radius of curvature of the implantable lead that bends along the inner transition region.

In Example 17, the implantable system according to Example 16, wherein at least a portion of the tubular body of the suture sleeve comprises silicone or polyurethane.

In Example 18, the implantable system according to Example 16 or 17, wherein the suture sleeve further comprises an anchor receiving area along the tubular body, the anchor receiving area defining an exterior surface within a channel that extends around a circumference of the tubular body.

In Example 19, the implantable system according to according to any of Examples 16-18, wherein the suture sleeve further comprises a protruding lip feature along the exterior surface of the tubular body at the distal end portion or the proximal end portion.

In Example 20, a suture sleeve for securing an implantable lead with a suture includes an elongate, tubular body having a proximal end portion with a proximal face, a distal end portion with a distal face, and an exterior surface. The suture sleeve also includes a lumen within the tubular body extending from the proximal end portion to the distal end portion, wherein the lumen is sized to receive the implantable lead. The suture sleeve also includes a nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control the radius of curvature of the implantable lead that bends along the inner transition region.

In Example 21, the suture sleeve according to Example 20, wherein the suture sleeve further comprises an outer transition region that extends between the exterior surface of the suture sleeve and at least one of the proximal face and the distal face of the suture sleeve.

In Example 22, the suture sleeve according to Example 20 or 21, wherein the inner transition region further comprises multiple curvilinear surfaces.

In Example 23, the suture sleeve according to according to any of Examples 20-22, wherein the inner transition region further comprises two or more chamfered edges, and wherein each chamfered edge has an obtuse angle between adjacent surfaces thereof.

In Example 24, the suture sleeve according to according to any of Examples 20-23, wherein the suture sleeve further comprises the inner transition region extending from the lumen to the proximal face of the tubular body.

In Example 25, the suture sleeve according to according to any of Examples 20-24, wherein the suture sleeve further comprises a second inner transition region extending from the lumen to the proximal face of the tubular body.

In Example 26, the suture sleeve according to according to Example 25, wherein the suture sleeve further comprises a second inner transition region extending from the lumen to the proximal end of the tubular body.

In Example 27, the suture sleeve according to according to any of Examples 20-26, wherein the suture sleeve further comprises the inner transition region that controls bending of the lead to a radius of curvature in a range of about 0.010 inches to 0.250 inches.

In Example 28, the suture sleeve according to according to any of Examples 20-26, wherein the suture sleeve further comprises at least a portion of the tubular body made of a first material and at least a portion of the tubular body that includes the inner transition region made of a second material having a higher durometer than the first material.

In Example 29, the suture sleeve according to according to Example 28, wherein the first material comprises silicone or polyurethane.

In Example 30, the suture sleeve according to according to Example 28, wherein the second material comprises a polyurethane, tecothane, polyether ether ketone (PEEK), stainless steel, MP35N, titanium and platinum.

In Example 31, a method of making a suture sleeve for securing an implantable lead includes forming a tubular body having a proximal end portion with a proximal face, a distal end portion with a distal face, an exterior surface, a lumen sized to receive the implantable lead and an inner surface within the lumen. The method also includes forming a nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control a radius of curvature of the implantable lead that bends along the inner transition region.

In Example 32, the method according to Example 31, wherein forming the tubular body includes forming a channel around the circumference of the tubular body, wherein the channel is sized to accommodate a suture within the channel.

In Example 33, the method according to Example 31 or 32, wherein forming the tubular body comprises using an injection molding process or an extrusion process.

In Example 34, the method according to according to any of Examples 31-33, wherein forming the inner transition region comprises using one of an injection molding, extrusion, sanding or a machining process.

In Example 35, the method according to according to any of Examples 31-34, wherein forming the tubular body includes forming the tubular body of silicone or polyurethane.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an implantable medical system including a suture sleeve, according to various embodiments.

FIG. 2A is a schematic illustration of the suture sleeve of FIG. 1.

FIGS. 2B-D are side, front and top views of the suture sleeve of FIG. 2A with some interior features shown to facilitate understanding.

FIG. 3 is a schematic illustration of portions of an alternative embodiment of the suture sleeve of FIG. 1.

FIG. 4 is a schematic illustration of portions of an alternative embodiment of the suture sleeve of FIG. 1.

FIG. 5 is a schematic illustration of portions of an alternative embodiment of the suture sleeve of FIG. 1.

FIG. 6 is a schematic illustration of portions of an alternative embodiment of the suture sleeve of FIG. 1.

FIG. 7 is a schematic illustration of portions of an alternative embodiment of the suture sleeve of FIG. 1.

FIG. 8 is a schematic illustration of portions of an alternative embodiment of the suture sleeve of FIG. 1.

While the invention is amenable to various modifications and alternative forms, specific embodiments have been shown by way of example in the drawings and are described in detail below. The intention, however, is not to control the invention to the particular embodiments described. On the contrary, the invention is intended to cover all modifications, equivalents, and alternatives falling within the scope of the invention as defined by the appended claims.

### DETAILED DESCRIPTION

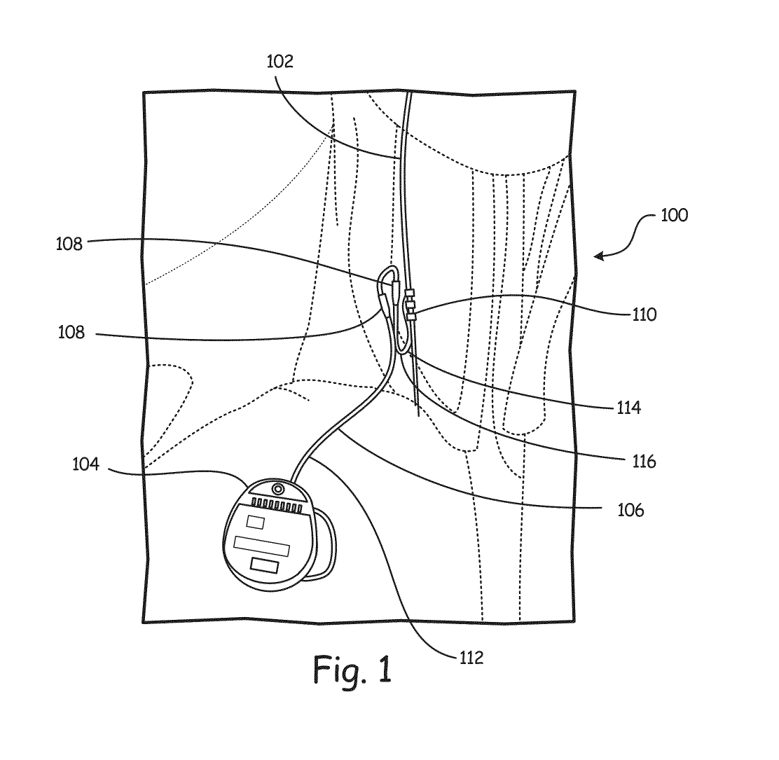

FIG. 1 is a simplified view of an implanted medical system 100 for stimulating a region of a patient's vagus nerve 102. The vagus nerve 102 is located within a carotid sheath (not shown) that consists of multiple layers of fascia wrapping the common carotid artery, the internal jugular vein (IJV), and the vagus nerve 102. As shown, the system 100 includes a pulse generator 104 implanted within a patient that is arranged for delivering electrical stimulation to the vagus nerve 102. The pulse generator 104 is typically implanted subcutaneously within an implantation location or pocket in the patient's chest or abdomen. The pulse generator 104 is connected to an implantable lead 106. The lead 106 operates to convey electrical signals between the implantable pulse generator 104 and the vagus nerve 102. The lead 106 includes a flexible lead body having a proximal end portion 112 and a distal end portion 114. In various embodiments, the lead 106 is coupled to the vagus nerve 102 by a connector 110, for example, a lead cuff or an insulating sheath. The lead connector 110 is disposed about and surrounds the carotid sheath or the vagus nerve 102, in some embodiments. In various embodiments, the system 100 can be used to selectively stimulate the vagus nerve 102, e.g. for treating cardiac disease. The lead 106 or the lead connector 110 can include electrodes (not shown in FIG. 1) that are electrically and operatively coupled to electronics and the power supply of the pulse generator 104 to electrically stimulate the vagus nerve 102 when implanted.

The proximal end portion 112 of the lead 106 can extend subcutaneously or submuscularly from the pulse generator 104 to the vascular entry site. At least a portion of the lead 106 can be secured by a securing means, such as suturing the lead 106 to subcutaneous tissue. The lead 106 can be secured by one or more sutures extending around the body of the lead 106 at a location proximal to the vascular entry site. In particular, the one or more sutures can wrap around a suture sleeve 108, also described as a lead anchor, which extends around the body of the lead 106, as will be discussed further herein.

Generally, suture sleeves 108 are used to secure the implantable lead 106 to body tissue to minimize chronic loading, movement and migration of the electrode. Suture sleeves 108 may also be used to secure a formation of a strain relief loop 116 to prevent loading to the electrode or delicate tissues and structures within the patient's body to minimize potential tissue damage or to reduce patient pain. For example, the formation of the strain relief loop 116 is commonly used in non-cardiac applications such as cervical vagus nerve stimulation or carotid sinus stimulation to prevent loading the nerves or carotid artery.

Although the lead 106 shown in FIG. 1 includes two suture sleeves 108, additional embodiments can include one or more than two suture sleeves 108 to help secure and stabilize the position of the lead 106 to surrounding musculature within the patient. The suture sleeve 108 may be positioned about the lead 106 proximal to and proximate the electrodes and/or distal to and proximate the pulse generator 104 in order to prevent longitudinal movement of these components. Although the pulse generator 104, the lead 106, and the suture sleeve 108 are illustrated in an arrangement for providing therapy to the vagus nerve 102, such components can be provided for sensing and/or stimulating various other organs, areas, nerves, or musculature of the body.

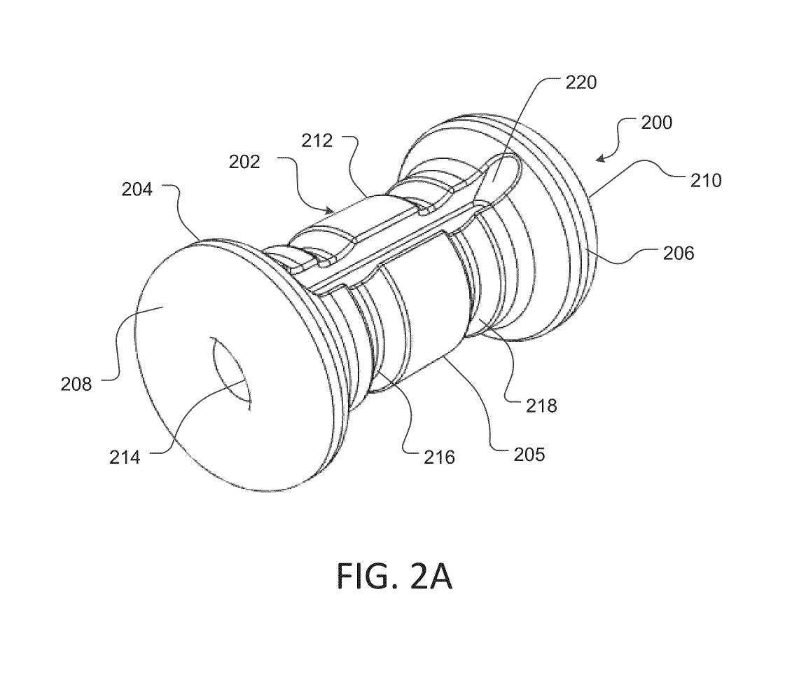

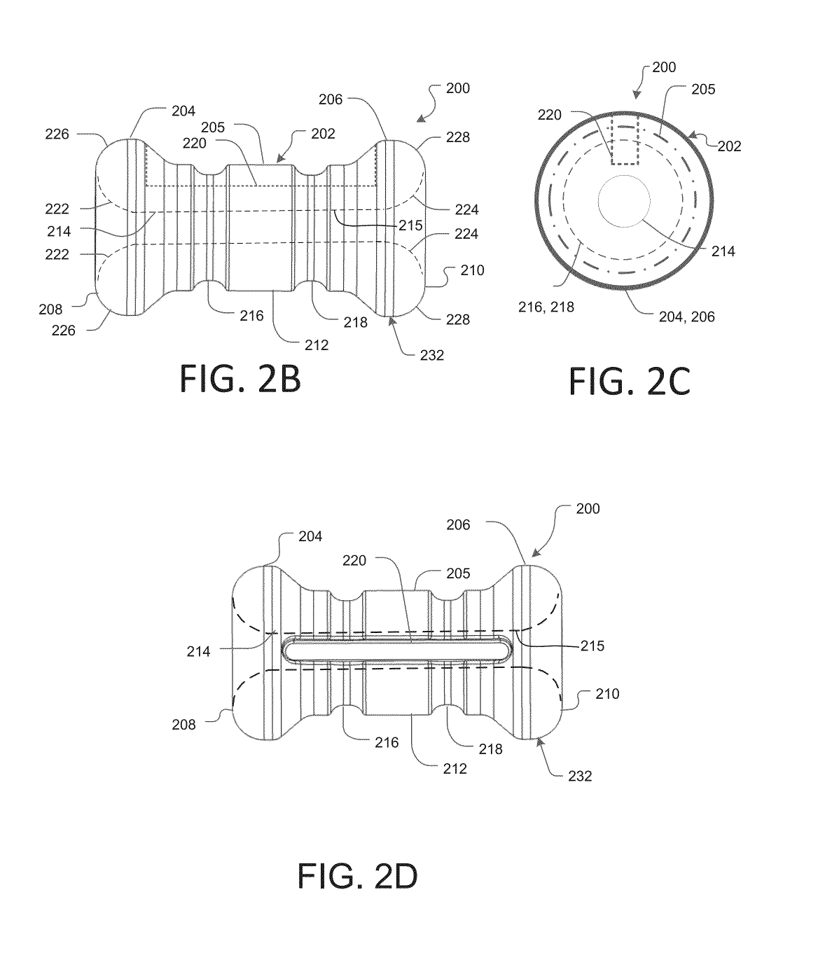

FIGS. 2A-2C are schematic illustrations of a suture sleeve 200, according to various embodiments. As shown in FIG. 2A, the suture sleeve 200 includes an elongate, hollow and tubular body 202. The tubular body 202 has a proximal end portion 204, a middle portion 205, and a distal end portion 206. As shown, the proximal end 204 portion has a proximal face 208, and the distal end portion 206 has a distal face 210. An exterior surface 212 of the tubular body 202 extends from the proximal face 208 to the distal face 210. The tubular body 202 includes a lumen 214 with an inner surface 215 within the tubular body 202 extending from the proximal end portion 204 to the distal end portion 206. In use, the lead 106 (FIG. 1) extends through the lumen 214 of the suture sleeve 200. As such, the lumen 214 is sized and shaped to receive the implantable lead 106.

The suture sleeve 200 may include at least one anchor receiving area 216, 218 along the tubular body 202, according to various embodiments. As shown in FIG. 2A, the suture sleeve 200 can include two anchor receiving areas 216, 218, for example, a proximal anchor receiving area 216 located at the proximal end portion 204 and a distal anchor receiving area 218 located at the distal end portion 206. Each of the proximal and distal anchor receiving areas 216, 218 can comprise a channel that extends transversely around the circumference of the suture sleeve 200. Reception of an anchoring element or anchoring means, such as sutures, within the channels can prevent the anchoring means from slipping off of the suture sleeve 200. Although FIG. 2A shows a tubular body 202 having two anchor receiving areas 216, 218, additional embodiments can include one or more than two anchor receiving areas 216, 218.

A plurality of anchoring means, such as sutures, can extend around the suture sleeve 200 and engage tissue, in some embodiments. Each suture can comprise a flexible string that can be wrapped around the suture sleeve 200 and threaded through tissue at an implant site. A knot can be tied in each suture after the suture is firmly wrapped around the suture sleeve 200 and thread through tissue at an implant site to anchor the suture sleeve 200 and the lead 106 (FIG. 1) to the implant site. The suture sleeve 200 can prevent the sutures from contacting the lead 106 to prevent the sutures from damaging the lead 106 while also securing the lead 106 at an implant site.

In various embodiments, the suture sleeve 200 may include at least one slot 220 formed in the tubular body 202. In some embodiments, the slot 220 extends longitudinally along at least a portion of the anchoring device. In some embodiments, the slot 220 extends longitudinally along an entire length of the tubular body 202, from the proximal end portion 204 to the distal end portion 206. The slot 220 facilitates compression of the tubular body 202 of the suture sleeve 200 under the tying force of the sutures. Described differently, the slot 220 allows the tubular body 202 to collapse inwardly when the sutures are tightened about the body 202. As shown in FIG. 2B, the slot 220 may extend partially inwardly into the tubular body 202, but without intersecting the inner lumen 214 of the tubular body 202. In other embodiments, the slot 220 extends partially into the tubular body 202 and intersects with the inner lumen 214. In some embodiments, the slot 220 extends through the tubular body 202 from the exterior surface 212 of a first side of the body 202 to the exterior surface 212 of a second, opposite side of the tubular body 202. Compression slots 220 are generally described in U.S. patent Ser. No. 12/265,285 filed Nov. 5, 2008, entitled COMPRESSION CONTROL LEAD ANCHORING DEVICE, which is incorporated herein by reference in its entirety.

FIGS. 2B-2D show side, front and top views of the suture sleeve 200 of FIG. 2A with some interior features shown to facilitate understanding. The tubular body 202 has at least one inner transition region 222, 224 (also described as a suture sleeve edge, a bearing surface or a radius feature) that defines a transition between the inner surface 215 and one face of the suture sleeve 200. Generally, a transition region defines a surface transition from a first surface to a second surface that is generally orthogonal to the first surface. In various embodiments, the inner transition region 222, 224 has a non-linear surface profile that extends from the inner surface 215 of the inner lumen 214 to one of the faces 208, 210 of the tubular body 202.

Described differently, in some embodiments, the inner transition region 222, 224 is a curve that connects two orthogonal surfaces at an angle other than a right angle. The curve may be defined as a straight curve or a radiused curve. For example, the inner transition region 222, 224 may be a curvilinear surface (or also described as a convex radius edge or rounded edge) having a suitable radius of curvature adapted to control the radius of curvature of the lead 106 of FIG. 1 bending against the inner transition region 222, 224, in some embodiments. As shown in FIGS. 2B and 2D, the inner transition region 222, 224 is a curved transition (may also be described as a curved edge or curved profile) extending between the inner lumen 214 and one of the faces 208, 210 of the tubular body 202. In other embodiments, the inner transition region 222, 224 is a chamfered edge having at least one obtuse angle between adjacent surfaces thereof, wherein the chamfered edge is adapted to control the radius of curvature of the lead 106 bending against the inner transition region 222, 224.

The inner transition region 222, 224 optionally comprises multiple obtuse angles or rounded curves. In some embodiments, the multiple non-linear curves are successive, connected regions. In other embodiments, the multiple non-linear curves are discrete, separate regions.

A benefit of using the suture sleeve 200 featuring an inner transition region 222, 224 includes eliminating a relatively sharp edge between the inner surface 215 and the adjacent face of the suture sleeve 200 to help minimize kinking of the lead 106 of FIG. 1. The inner transition region 222, 224 is adapted to create a non-linear surface that increases the radius of curvature of the lead 106 as it bends or curls against the inner transition region 222, 224. The inner transition region 222, 224 is configured to control the radius of curvature of the lead 106, as desired. In some embodiments, the inner transition region 222, 224 is configured to control the radius of curvature of the lead 106 to a radius that is greater than the kink radius of the lead 106, i.e. a radius that would likely cause the lead body to kink.

Another benefit of using a suture sleeve 200 with a transition region is to minimize damaging the exterior surface 212 of the lead body when the lead body rubs against the inner edges of the suture sleeve 200. The curvilinear surface is the preferred embodiment for minimizing surface damage that might be caused by interaction between the suture sleeve inner edges and the lead body.

In various embodiments, the suture sleeve 200 has one or two inner transition regions 222, 224. For example, in FIG. 2B, the suture sleeve 200 includes a proximal inner transition region 222 at the proximal end region 204 and a distal inner transition region 224 at the distal end region 206. As shown, the proximal inner transition region 222 extends from the inner surface 215 of the lumen 214 to the proximal face 208 and the distal inner transition region 224 extends from the inner surface 215 of the lumen 214 to the distal face 210. In some embodiments, the suture sleeve 200 includes one transition region at one end portion, e.g., the proximal end portion 204. Alternatively, in other embodiments, the suture sleeve 200 includes one transition region at the distal end portion 206 only. As discussed previously herein, the inner transition region 222, 224 is configured to control the radius of curvature of the implantable lead 106 as it bends along the surface of the inner transition region 222, 224.

The inner transition region 222, 224 may generally form a radius of curvature in a range of about 0.010 inches to 0.250 inches, for example. In some embodiments, the radius of curvature formed may be about 0.062 inches, for example. Other examples of suitable radii of curvature ranges may include about 0.010 inches to 0.020 inches, about 0.020 inches to 0.025 inches or about 0.025 inches to 0.065 inches. In still further suitable examples, the radius of curvature range may be about 0.065 inches to about 0.100 inches, about 0.1 inches to 0.2 inches or about 0.20 inches to 0.25 inches. In some embodiments, a suitable radius of curvature for the suture sleeve 200 may be any radius greater than the minimum bend radius of the implantable lead body portion that interacts with the suture sleeve 200.

In some embodiments, the suture sleeve 200 includes an outer transition region 226, 228 along at least a portion of the exterior surface 212, in some embodiments. The outer transition region 226, 228 may share features that are similar to the inner transition portion, except that the outer transition region 226, 228 defines a transition located between the exterior surface 212 of the suture sleeve 200 and at least one face of the suture sleeve 200. In various embodiments, the outer transition region 226, 228 is a non-linear profile that extends from the exterior surface 212 of the tubular body 202 to one of the faces 208, 210 of the tubular body 202.

As shown in FIGS. 2B and 2D, the outer transition region 226, 228 is a curved transition extending between the exterior surface 212 and one of the faces 208, 210 of the tubular body 202. Similar to the inner transition region 222, 224, the outer transition region 226, 228 may include at least one curvilinear surface having a suitable radius of curvature that extends from the exterior surface 212 of the tubular body 202 to one of the faces 208, 210, in some embodiments. In other embodiments, the outer transition region 226, 228 may include at least one chamfered edge at an angle that is either obtuse to adjacent surfaces thereof or to the exterior surface 212 or one of the faces of the suture sleeve 200. The outer transition region is adapted to control the radius of curvature of the lead 106 of FIG. 1 curled against at least a portion of the external region of the suture sleeve 200, in some embodiments.

The outer transition region 226, 228 may comprise a curve that extends smoothly and continuously from the inner transition region 222, 224, in some embodiments, as illustrated in FIG. 2B. In other embodiments, the outer transition region 226, 228 and the inner transition regions 222, 224 may be discontinuous, separate regions that are separated by a portion of one of the faces.

In FIG. 2B, the suture sleeve 200 includes inner transition regions 222, 224 and the outer transition regions 226, 228 having dimensionally equivalent or similar radii of curvatures. In other embodiments, the inner transition portion may have a larger or smaller radius of curvature than the outer transition portion.

In various embodiments, the inner transition region 222, 224 or the outer transition region 226, 228 comprises multiple curvilinear surfaces, wherein each curvilinear surface has a specific radius of curvature. For example, in some embodiments, the multiple curvilinear surfaces of the outer transition region 226, 228 can have varying radii of curvature. Alternatively, in some embodiments, the multiple curved transitions can have equivalent or similar radii of curvature.

Suitable materials for the suture sleeve 200 include various polymeric, ceramic and metallic materials. Some examples of suitable polymer materials for the suture sleeve 200 include, but are not limited to, silicone, polyurethane, polycarbonate, tecothane, polyether, polyester, polyisobutylene (PIB) polyurethane, polyamide, nylon 6, nylon 12, polyetherimide and/or combinations thereof. Some examples of suitable metallic materials include, but are not limited to, stainless steel, MP35N titanium, platinum and/or combinations thereof.

In various embodiments, the suture sleeve 200 comprises multiple suitable materials including various polymeric, ceramic and metallic materials. In some embodiments, at least a portion of the suture sleeve 200 is formed from a first material and a second material. The second material, in various embodiments, has different material or mechanical properties than the first material, e.g., durometer, tensile strength, and lubricity. In some embodiments, an inner portion of the suture sleeve 200 may be made of the first material, while an outer portion of the suture sleeve 200 is made of the second material. For example, in some embodiments, a portion of the tubular body 202 of the suture sleeve 200 comprising the exterior surface 212 can be made a low durometer material, e.g., silicone, while a portion of the tubular body 202 that includes the inner transition region 222, 224 can be made of a second material with high durometer, e.g. PEEK.

FIGS. 2-5 provide perspective views of alternative embodiments of the suture sleeve 108 from FIG. 1 that includes a protruding lip feature 232, 332, 432, 532, according to various embodiments.

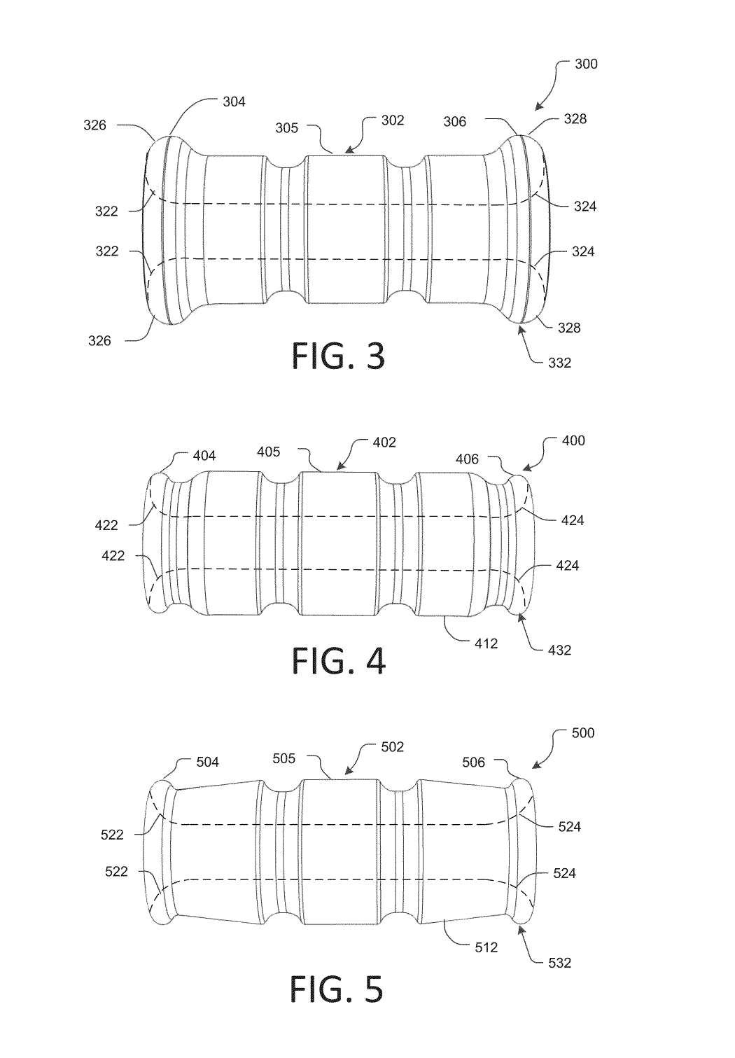

FIG. 3 is a perspective view of alternative embodiments of a suture sleeve 300 with a tubular body 302. As shown, the tubular body 302 of the suture sleeve 300 has an outer diameter that varies along the length of the tubular body 302. In various embodiments, the suture sleeve 300 has a protruding lip feature 332 at each of the end portions 304, 306. In some embodiments, the tubular body 302 has the protruding lip feature 332 at only one end portion of the body (not shown), for example, the proximal end portion 304. The protruding lip feature 332 is defined by an increased outer diameter at the distal end portion 306 and/or the proximal end portion 304 that is larger than the outer diameter at the middle portion 305 of the suture sleeve 300. The outer diameter of the protruding lip feature 332 defines a maximum diameter of the suture sleeve 300, in some embodiments. The protruding lip feature 332 optionally tapers to a smaller diameter at a location more proximate to the middle portion 305 of the suture sleeve 300. In some embodiments, the protruding feature diameter tapers to a diameter that is equivalent or similar to the outer diameter of the middle portion 305 of the suture sleeve 300. In other embodiments, the protruding feature diameter tapers to a diameter that is larger than the outer diameter at the middle portion 305 of the suture sleeve 300.

Furthermore, as shown in FIG. 3, the suture sleeve 300 has an outer transition region 326, 328 with a different radius of curvature than an inner transition region 322,324. More specifically, as shown, the inner transition profile has a larger radius of curvature than the outer transition region 326, 328. In some embodiments, the inner transition region 322,324 may, however, have a smaller radius of curvature than the outer transition region 326, 328.

FIG. 4 provides perspective views of alternative embodiments of a suture sleeve 400 that includes a tubular body 402 having a protruding lip feature 432. In some embodiments, the protruding lip feature 432 has an outer diameter that is similar or equivalent to an outer diameter of the suture sleeve 400 at a middle portion 405. As such, in some embodiments, the inclusion of the protruding lip feature 432 does not increase the maximum outer diameter of the suture sleeve 400. In some embodiments, the outer diameter of the protruding lip feature 432 at the proximal and/or distal end portion 404, 406 of the suture sleeve 400 may be equal to or smaller than an outer diameter of the middle portion 405 of the tubular body 402.

FIG. 5 provides perspective views of alternative embodiments of a suture sleeve 500 that includes a tubular body 502 having a protruding lip feature 532. As shown, the protruding lip feature 532 has an outer diameter that is similar or equivalent to the outer diameter of the suture sleeve 400 at the middle portion 505.

As shown in FIGS. 4 and 5, the protruding lip feature 432, 532 is defined by an outer transitional region extending from the proximal face or distal face of the suture sleeve 400, 500 and a depressed region extending from the middle portion 405, 505 of the tubular body 502. In some embodiments, the depressed region has a diameter that is smaller than the middle portion 405, 505 or the protruding lip feature 432, 532 of the suture sleeve 400, 500. In some embodiments, a portion of the depressed region proximate to the middle portion 405, 505 sharply increases to a diameter that is equivalent to or similar to the outer diameter at the middle portion 405, 505, such that the depressed region forms a groove or a channel proximate to the protruding lip feature 432, 532. In other embodiments, a side of the depressed region facing the middle portion 405, 505 gradually increases to an outer diameter that is equivalent to or similar to the outer diameter of the middle portion 405, 505 such that the depressed region defines a tapered region that extends from the middle portion 405, 505 to the proximal end portion 404, 504 or the distal end portion 406, 506.

The protruding lip feature 432, 532 may allow for the increase or decrease of a maximum radius of curvature of an inner transition region 422, 424, 522, 524. In some embodiments, the protruding lip feature 432, 532 having an outer diameter that is larger than the nominal outer diameter (i.e. the outer diameter at the middle portion 405, 505) allows for a larger maximum radius of curvature of the inner transition region 422, 424, 522, 524. In other embodiments, the protruding lip feature 432, 532 having an outer diameter that is smaller than the nominal outer diameter reduces or limits the maximum radius of curvature of the inner transition region 422, 424, 522, 524.

The protruding lip feature 432, 532 can provide several benefits for medical electric leads. The protruding lip feature 432, 532 may provide support to and control the bend radius of the medical lead 106 that is bending or curling along the exterior surface 412, 512 of the suture sleeve 400. In some embodiments, the protruding lip feature can provide support to a lead 106 bending greater than about 90 degrees. For example, the suture sleeve 400 with the protruding lip feature 432, 532 may provide support to the medical lead 106 bending between about 90 degrees and 180 degrees or between about 90 degrees and about 120 degrees. The term “about” may be used to define an angle value that is within 1 to 5 degrees from the specified value.

Other benefits of using a suture sleeve 400, 500 with the protruding lip feature 432, 532 include providing an outer transition region 426, 428, 526, 528, that minimizes an abrasive edge that might damage the exterior surface 412, 512 of the lead body when the lead body rubs against the exterior surface 412, 512 of the protruding lip feature 432, 532.

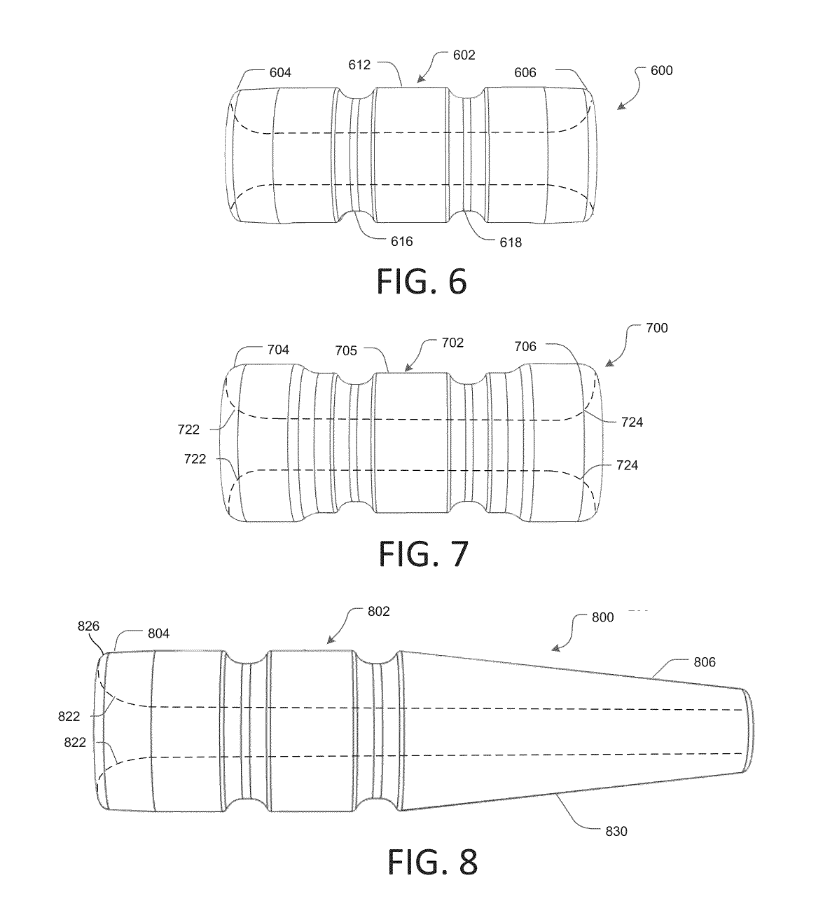

FIG. 6 is a perspective view of an alternative suture sleeve 600 with a tubular body 602 having distal and proximal end portions 606, 604. In the embodiment shown, the suture sleeve 600 has a generally uniform outer diameter along an entire exterior surface 612 of the tubular body 602 from the proximal end portion 604 to the distal end portion 606, with the exception of a proximal anchor-receiving area 616 and a distal anchor-receiving area 618.

FIG. 7 shows an embodiment of an alternative suture sleeve 700 with a tubular body 702 having an enlarged, uniform outer diameter extending from either the proximal end portion 704 or the distal end portion 706 towards a middle portion 705 of the suture sleeve 700. As shown, in some embodiments, the enlarged diameter tapers to a smaller diameter at a location proximate to the middle portion 705. In some embodiments, the enlarged diameter may decrease to a smaller outer diameter at a location proximate to the middle portion 705 that is equivalent or similar to the diameter of the middle portion 705. In some embodiments, the smaller diameter at the middle portion 705 of the body extends between the proximal and the distal end portions 704, 706. The enlarged diameter at the proximal and distal end portions 704, 706 may facilitate increasing the radius of curvature of the inner transition region 722, 724 while the smaller diameter near and at the middle portion 705 helps to decrease the amount of tissue displacement caused by the suture sleeve 600 at the implant site.

FIG. 8 shows an alternative embodiment of the suture sleeve 800 with a tubular body 802 having a tapered end section 830 at a distal end portion 806 to allow for easier insertion into a patient's blood vessel. As further shown in FIG. 8, the suture sleeve 800 includes an outer transition region 826 at a proximal end portion 804 of the tubular body 802, but no outer transition region at the tapered end section 830 at the distal end portion 806. In some embodiments, the radius of curvature of the outer transition region 826 at one end of the suture sleeve 800 may be smaller or larger than the radius of curvature of the outer transition region at the opposite end (not shown). For example, the outer transition region 826 at the proximal end portion 804 can have a larger or smaller radius of curvature than the outer transition region at the distal end portion 806, in some embodiments.

Similarly, the suture sleeve 800 includes an inner transition region 822 at the proximal end portion 804 of the tubular body 802, but no inner transition region at the distal end portion 806. In some embodiments, the inner transition region 822 at the proximal end portion 804 can be larger or smaller than the radii or curved profile of the inner transition region at the distal end portion 806.

The various embodiments of the suture sleeves described herein can be made using a range of manufacturing techniques. For example, one method of making a suture sleeve comprises forming the tubular body that includes the proximal end portion with the proximal end and the distal end portion with the distal end, in some embodiments. The tubular body also includes the exterior surface and the lumen with an inner surface, wherein the lumen is sized to receive the implantable lead. In some embodiments, forming a tubular body includes forming a channel around the circumference of the tubular body, wherein the channel is sized to accommodate a suture within the channel. In some embodiments, forming the tubular body comprises using an injection molding process or an extrusion process.

The method of making a suture sleeve further comprising forming the nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control a radius of curvature of the implantable lead that bends along the inner transition region, in some embodiments. In some embodiments, forming the inner transition region comprises using one of an injection molding, extrusion, sanding or a machining process.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations that fall within the scope of the claims, together with all equivalents thereof.

We claim:

1. An implantable system comprising:
a pulse generator;
an implantable medical electrical lead having a lead body with a proximal end and a distal end, the proximal end coupled to the pulse generator; and
a suture sleeve for securing an implantable lead with a suture, the suture sleeve comprising:
an elongate, tubular body having a proximal end portion with a proximal face, a distal end portion with a distal face, and an exterior surface;
a lumen having an inner surface within the tubular body that extends from the proximal end portion to the distal end portion, the lumen sized to receive the implantable lead;
a non-linear, inner transition region extending from the lumen to at least one of the proximal face and the distal face of the tubular body, the inner transition region being a curvilinear surface or a chamfered edge having an obtuse angle between adjacent surfaces thereof, wherein the inner transition region is adapted to control a radius of curvature of the implantable lead that bends along the inner transition region;
at least one anchoring element configured to be secured circumferentially about the tubular body to secure the suture sleeve at particular location within the patient's body.

2. The implantable system of claim 1, wherein at least a portion of the tubular body of the suture sleeve comprises silicone or polyurethane.

3. The implantable system of claim 1, wherein the suture sleeve further comprises an anchor receiving area along the tubular body, the anchor receiving area defining an exterior surface within a channel that extends around a circumference of the tubular body.

4. The implantable system of claim 1, wherein the suture sleeve further comprises a protruding lip feature along the exterior surface of the tubular body at the distal end portion or the proximal end portion.

5. A suture sleeve for securing an implantable lead with a suture, the suture sleeve comprising:
an elongate, tubular body having a proximal end portion with a proximal face, a distal end portion with a distal face, and an exterior surface;
a lumen within the tubular body extending from the proximal end portion to the distal end portion, the lumen sized to receive the implantable lead;
a nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control a radius of curvature of the implantable lead that bends along the inner transition region.

6. The suture sleeve of claim 5, wherein the suture sleeve further comprises an outer transition region that extends between the exterior surface of the suture sleeve and at least one of the proximal face and the distal face of the suture sleeve.

7. The suture sleeve of claim 5, wherein the inner transition region further comprises multiple curvilinear surfaces.

8. The suture sleeve of claim 5, wherein the inner transition region further comprises two or more chamfered edges, and wherein each chamfered edge has an obtuse angle between adjacent surfaces thereof.

9. The suture sleeve of claim 5, wherein the suture sleeve further comprises the inner transition region extending from the lumen to the proximal face of the tubular body.

10. The suture sleeve of claim 5, wherein the suture sleeve further comprises the inner transition region extending from the lumen to the distal face of the tubular body.

11. The suture sleeve of claim 10, wherein the suture sleeve further comprises a second inner transition region extending from the lumen to the proximal face of the tubular body.

12. The suture sleeve of claim 5, wherein the suture sleeve further comprises the inner transition region that controls bending of the lead to a radius of curvature in a range of about 0.010 inches to 0.250 inches.

13. The suture sleeve of claim 5, wherein the suture sleeve further comprises at least a portion of the tubular body made of a first material and at least a portion of the tubular body that includes the inner transition region made of a second material having a higher durometer than the first material.

14. The suture sleeve of claim 13, wherein the first material comprises silicone or polyurethane.

15. The suture sleeve of claim 13, wherein the second material comprises one of a polyurethane, tecothane, polyether ether ketone (PEEK), stainless steel, MP35N, titanium and platinum.

16. A method of making a suture sleeve for securing an implantable lead, the method comprising:
forming a tubular body having a proximal end portion with a proximal face, a distal end portion with a distal face, an exterior surface, a lumen sized to receive the implantable lead, an inner surface within the lumen; and
forming a nonlinear, inner transition region extending from the lumen to at least one of the proximal face and the distal face of the tubular body, wherein the inner transition region is a curvilinear surface or a chamfered edge having an obtuse angle between adjacent surfaces thereof and is adapted to control a radius of curvature of the implantable lead that bends along the inner transition region.

17. The method of claim 16, wherein forming the tubular body includes forming a channel around the circumference of the tubular body, wherein the channel is sized to accommodate a suture within the channel.

18. The method of claim 16, wherein forming the tubular body comprises using an injection molding process or an extrusion process.

19. The method of claim 16, wherein forming the inner transition region comprises using one of an injection molding, extrusion, sanding or a machining process.

20. The method of claim 16, wherein forming the tubular body includes forming the tubular body of silicone or polyurethane.
